(19) United States
(12) Patent Application Publication
(10) Pub. No.: US 2024/0370938 A1
(43) Pub. Date: Nov. 7, 2024

(54) METHOD, SYSTEM AND PROGRAM FOR DETERMINING INSURANCE RATE FOR PET BY USING AT LEAST ONE INTERNET OF THINGS (IOT) DEVICE USED IN DAILY LIFE OF PET

(71) Applicant: PurrSong, Inc., Daejeon (KR)

(72) Inventors: Tae Gu NOH, Suwon-si Gyeonggi-do (KR); Chang Hyeon BAK, Goyang-si Gyeonggi-do (KR)

(73) Assignee: PurrSong, Inc., Daejeon (KR)

(21) Appl. No.: 18/773,671

(22) Filed: Jul. 16, 2024

Related U.S. Application Data
(63) Continuation of application No. PCT/KR2024/003102, filed on Mar. 11, 2024.

(30) Foreign Application Priority Data
Mar. 10, 2023 (KR) 10-2023-0031533

(51) Int. Cl. G06Q 40/08; G16H 50/30

(52) U.S. Cl. CPC G06Q 40/08; G16H 50/30

(57) ABSTRACT

A method for calculating insurance premiums for pets using at least one Internet of Things (IoT) device employed in the life of pets according to an embodiment of the present disclosure may include, collecting basic information of the pet, collecting maintenance information about maintenance of the at least one IoT device by a pet owner and usage information about usage of the at least one IoT device by the pet, calculating a level of conscientiousness of the pet owner based on the maintenance information, calculating a level of health of the pet based on the usage information, and determining the insurance rate for the pet based on the level of conscientiousness of the pet owner and the level of health of the pet.

### CROSS-REFERENCE TO RELATED APPLICATIONS

This application is a continuation of International Application No. PCT/KR2024/003102 filed on Mar. 11, 2024, which claims priority to Korean Patent Application No. 10-2023-0031533 filed on Mar. 10, 2023, the entire contents of which are herein incorporated by reference.

### TECHNICAL FIELD

The present disclosure relates to a method, system and program for calculating an insurance rate for a pet by using at least one Internet of Things (IoT) device used in the daily life of the pet.

### BACKGROUND

With the increasing popularity of pets, the number of households raising pets is increasing greatly. Accordingly, the pet-related expenditures and markets are also increasing, and the associated medical costs have increased greatly.

Pet insurance is one of the best methods for reducing the medical costs of a pet and ensuring the safety of the pet. However, the pet insurance has been underutilized to date. Such low utilization of the pet insurance may cause insurance fees to be improperly high.

In general, an insurance provider participates in the underwriting and assessing insurance for assessing the level of risk related to the asset to be insured and determining a proper insurance rate to protect the asset against the risk. A health insurance and vehicle insurance policy provider determines a proper insurance rate for a person by using a highly refined insurance statistical model based on reliable data from tens of millions of insurance companies, medical records, and police reports. Compared to this, an insurance rate for a pet may be determined without access to reliable data. In many cases, pet insurance providers lack effective means for checking the accuracy of data provided by pet owners.

Therefore, an insurance rate for a pet may have a relatively high uncertainty, which may cause an insurance provider to increase insurance fees for protection against financial loss.

### DISCLOSURE OF THE INVENTION

### Problems to be Solved by the Invention

The present disclosure is conceived to determine an insurance rate for a pet based on level of conscientiousness of a pet owner and a level of health of a pet in order to improve an unreasonable insurance rate for a pet.

The problems to be solved by the present disclosure are not limited to the above-described problems. There may be other problems to be solved by the present disclosure.

### Means for Solving the Problems

A method for calculating insurance premiums for pets using at least one Internet of Things (IoT) device employed in the life of pets according to an embodiment of the present disclosure may include, collecting basic information of the pet, collecting maintenance information about maintenance of the at least one IoT device by a pet owner and usage information about usage of the at least one IoT device by the pet, calculating a level of conscientiousness of the pet owner based on the maintenance information, calculating a level of health of the pet based on the usage information, and determining the insurance rate for the pet based on the level of conscientiousness of the pet owner and the level of health of the pet.

A system for calculating insurance premiums for pets using at least one IoT device employed in the life of pets according to an embodiment of the present disclosure may include an IoT device, and a data processor. The data processor includes, a collection unit configured to collect basic information of the pet, maintenance information about maintenance of the at least one IoT device by a pet owner, and usage information about usage of the at least one IoT device by the pet, a calculation unit configured to calculate a level of conscientiousness of the pet owner based on the maintenance information and calculate a level of health of the pet based on the usage information, and a determination unit configured to determine the insurance rate for the pet based on the level of conscientiousness of the pet owner and the level of health of the pet.

A computer program stored on a computer-readable recording medium for calculating insurance premiums for pets using at least one IoT device employed in the life of pets according to an embodiment of the present disclosure, when executed by a computing device, may causes the computing device to, collect basic information of the pet, collect maintenance information about maintenance of the at least one IoT device by a pet owner and usage information about usage of the at least one IoT device by the pet, calculate a level of conscientiousness of the pet owner based on the maintenance information, calculate a level of health of the pet based on the usage information, and determine the insurance rate for the pet based on the level of conscientiousness of the pet owner and the level of health of the pet.

### Effects of the Invention

According to an embodiment of present disclosure, an insurance provider can suppress financial loss by reducing the uncertainty about the risk of accident of a pet.

Also, an insurance rate reasonably calculated based on the risk of accident of a pet can be applied to an insurant.

### BRIEF DESCRIPTION OF THE DRAWINGS

FIG. 1 is a configuration diagram of a system for determining an insurance rate for a pet according to an embodiment of the present disclosure.

FIG. 2 is a flowchart showing a method for determining an insurance rate for a pet according to an embodiment of the present disclosure.

### BEST MODE FOR CARRYING OUT THE INVENTION

Hereinafter, embodiments of the present disclosure will be described in detail with reference to the accompanying drawings to be readily implemented by a person with ordinary skill in the art to which the present disclosure belongs. However, it is to be noted that the present disclosure is not limited to the example embodiments but can be embodied in various other ways. In the drawings, parts irrelevant to the description are omitted in order to clearly explain the present disclosure, and like reference numerals denote like parts through the whole specification.

Through the whole specification, when a member is said to be located “on” another member, this includes not only the case where the member is in contact with the other member, but also the case where another member exists between the two members.

Through the whole specification, when a part “comprises or includes” a certain component, this means that it may further include other components rather than excluding other components unless specifically stated to the contrary.

As used through the whole specification, the terms “about”, “substantially”, etc. are used to mean at or close to that value when manufacturing and material tolerances inherent to the stated meaning are presented, it is used to prevent unscrupulous infringers from taking unfair advantage of disclosures in which precise or absolute figures are mentioned to help understanding of the present disclosure. The term “step of” or “step of” as used through the whole specification does not mean “step for.”

Through the whole specification, the term “combination(s) thereof” included in the Markushi format expression means a mixture or combination of one or more selected from a group consisting of the components described in the Markushi format expression, and means containing one or more selected from the group consisting of the above components. Through the whole specification, references to “A and/or B” mean “A or B, or A and B.”

Hereinafter, embodiments of the present disclosure will be described in detail with reference to the accompanying drawings. However, the present disclosure may not be limited to these implementations, examples, and drawings.

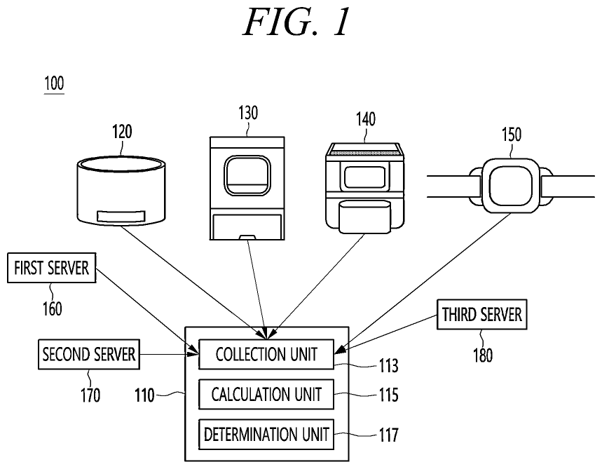

FIG. 1 is a configuration diagram of a system 100 for determining an insurance rate for a pet according to an embodiment of the present disclosure.

Referring to FIG. 1, the system 100 for determining an insurance rate for a pet according to an embodiment of the present disclosure may include a data processor 110, IoT devices 120, 130, 140 and 150, a first server 160, a second server 170 and/or a third server 180.

In an embodiment, each of the data processor 110, the IoT devices 120, 130, 140 and 150, the first server 160, the second server 170 and/or the third server 180 may include a communication module (not shown) configured to transmit and receive data according to a predetermined communication scheme (e.g., wireless communication, etc.).

The data processor 110 according to an embodiment may be a server host computer. In another embodiment, the data processor 110 may be any type of devices including a portable communication device (e.g., a smartphone), a computer device, a portable multimedia device, a portable medical device, a camera, an electronic device, a home appliance, a tablet terminal, a personal digital assistant (PDA), a desktop personal computer (PC), and a notebook PC. In an embodiment, the data processor 110 may include a collection unit 113, a calculation unit 115 and/or a determination unit 117.

In an embodiment, the collection unit 113 may be configured to collect basic information of a pet, maintenance information about maintenance of at least one of the IoT devices 120, 130, 140 and 150 by a pet owner, and usage information about usage of at least one of the IoT devices 120, 130, 140 and 150 by the pet.

In an embodiment, the calculation unit 115 may be configured to calculate a level of conscientiousness of the pet owner based on the maintenance information and calculate a level of health of the pet based on the usage information. For example, the calculation unit 115 may be trained to calculate the level of conscientiousness of the pet owner and/or the level of health of the pet based on the maintenance information and/or the usage information, respectively.

In an embodiment, the determination unit 117 may be configured to determine an insurance rate for the pet based on the level of conscientiousness of the pet owner and the level of health of the pet.

The data processor 110 according to an embodiment may be a computer readable storage medium that stores a computer program including a sequence of instructions for determining the insurance rate for the pet by using at least one of the IoT devices 120, 130, 140 and 150 used in the daily life of the pet.

An IoT (Internet of Things) device according to an embodiment is a device having a built-in wireless communication function, and may have a distinguishable Internet Protocol (IP) address. In an embodiment, the IoT devices 120, 130, 140 and 150 may be connected to a terminal device, a server and/or the Internet through wireless communication (e.g., WiFi or Bluetooth®), and may transmit and receive data to and from the device connected through wireless communication.

In an embodiment, the IoT devices 120, 130, 140 and 150 may be devices used in the daily life of the pet. For example, the IoT devices 120, 130, 140 and 150 may acquire the usage information about usage of the IoT devices 120, 130, 140 and 150 by the pet.

In an embodiment, the IoT devices 120, 130, 140 and 150 may include at least one of a toilet 140, a tracker 150, a feeder 130 or a water dispenser 120 for use by the pet.

In an embodiment, the water dispenser 120 serves to provide water to the pet, and may be configured to measure amount of water intake of the pet and acquire data about the measured amount of water intake.

In an embodiment, the feeder 130 serves to provide feed to the pet, and may be configured to measure amount of feed and/or nutrient intake of the pet and acquire data about the measured amount of feed and/or nutrient intake.

In an embodiment, the toilet 140 serves to collect feces of the pet, and may be configured to measure a usage pattern of the pet of the toilet 140 and acquire data about the measured usage pattern.

In an embodiment, the tracker 150 is mounted on the pet, and may be configured to detect a location, a behavior and/or a movement of the pet and acquire biometric data of the pet. The tracker 150 may be configured to measure amount of activity of the pet and acquire data about the measured amount of activity.

In an embodiment, the IoT devices 120, 130, 140 and 150 (e.g., the toilet 140 and the like) may be configured to measure a body weight of the pet and acquire information about the measured body weight.

The first server 160, the second server 170 and/or the third server 180 according to an embodiment may be a computer or a server, which is software, that provides a service or data to a client device in a network through the Internet. Herein, each of the first server 160, the second server 170 and/or the third server 180 may be an individual server, or at least some of the servers may be integrated with each other or may be integrated with the data processor 110.

In an embodiment, the first server 160 may be a management server or database that stores the basic information of the pet and input information input by the pet owner into an application corresponding to at least one of the IoT devices 120, 130, 140 and 150. In an embodiment, the first server 160 may be configured to receive, from a terminal device (not shown) of the pet owner, the basic information of the pet and input information input into an application corresponding to at least one of the IoT devices 120, 130, 140 and 150, and store the received information.

In an embodiment, the second server 170 may be a shopping mall server or database that stores purchase information of items for the pet.

In an embodiment, the third server 180 may be a medical data storage server or database that stores medical records of the pet.

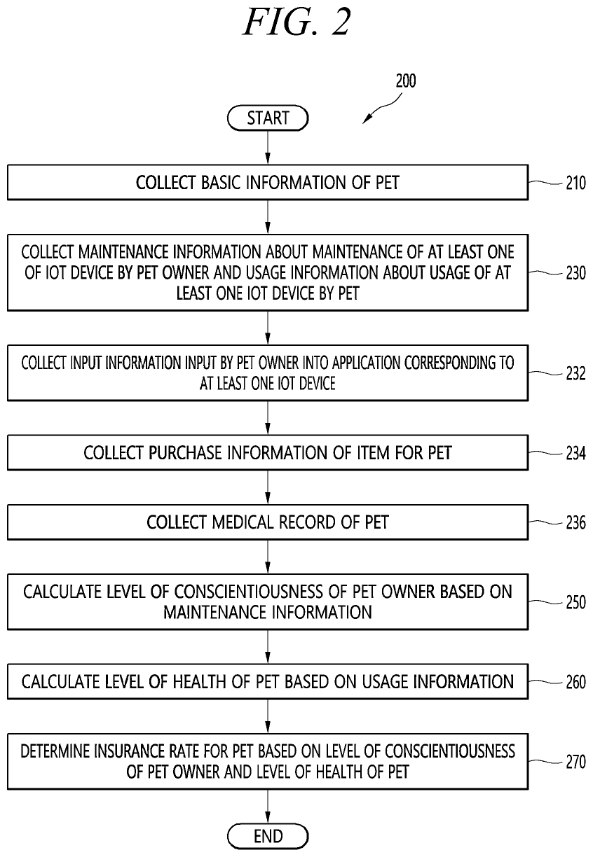

FIG. 2 is a flowchart 200 showing a method for determining an insurance rate for a pet according to an embodiment of the present disclosure.

Referring to FIG. 2, the data processor 110 according to an embodiment may collect the basic information of the pet in a process 210. In an embodiment, the data processor 110 may receive the basic information of the pet stored in the first server 160 from the first server 160.

For example, the pet may be plural in number, and a specific pet may be identified from among a plurality of pets based on photos, biometric data and/or an RFID chip.

In an embodiment, the data processor 110 may acquire the basic information of the pet stored in the first server 160 and collect information, which will be described later, about the pet corresponding to the acquired basic information.

In an embodiment, the basic information of the pet may include at least one of a breed, a photo, biometric data, an age, a height, a body weight or initial health conditions of the pet. For example, the basic information of the pet may further include daily habits, an initial medical examination result or a residential zone of the pet, or biographical information of the pet owner.

The data processor 110 according to an embodiment may collect the maintenance information about maintenance of at least one of the IoT devices 120, 130, 140 and 150 by the pet owner and the usage information about usage of at least one of the IoT devices 120, 130, 140 and 150 by the pet in a process 230.

In an embodiment, the data processor 110 may acquire the maintenance information and/or the usage information of at least one of the IoT devices 120, 130, 140 and 150 from the at least one of the IoT devices 120, 130, 140 and 150 and/or the first server 160. For example, the data processor 110 may acquire the maintenance information of at least one of the IoT devices 120, 130, 140 and 150 from a sensor included in the at least one of the IoT devices 120, 130, 140 and 150. For example, the data processor 110 may acquire, from the first server 160, the maintenance information input by the pet owner into an application.

For example, the maintenance information of the IoT devices 120, 130, 140 and 150 may include at least one of cleaning information, consumable replacement information, water supply information or feed supply information of at least one of the IoT devices 120, 130, 140 and 150.

For example, the usage information of the IoT devices 120, 130, 140 and 150 may include at least one of usage of the toilet, the amount of activity, the amount of feed intake or the amount of water intake of the pet.

The data processor 110 according to an embodiment may collect the input information input by the pet owner into an application corresponding to at least one of the IoT devices 120, 130, 140 and 150 in a process 232.

In an embodiment, the data processor 110 may receive, from the first server 160, the input information input by the pet owner into an application corresponding to at least one of the IoT devices 120, 130, 140 and 150 and stored in the first server 160.

For example, the input information may be a management log input by the pet owner into an application and stored in the first server 160, and the management log may contain information about activity (e.g., walk, exercise, play) with the pet, feed supply, claw care, and the like.

The data processor 110 according to an embodiment may collect the purchase information of the items for the pet in a process 234.

In an embodiment, the data processor 110 may collect the purchase information of the items for the pet from payment history at an exclusive shopping mall for the pet or of an exclusive payment card stored in the second server 170.

For example, the purchase information may include the kind of an item for the pet, the amount of purchase, the frequency of purchase or purchase costs.

For example, the items for the pet may include at least one of feeds, nutrients, joint braces, filters or consumables.

The data processor 110 according to an embodiment may collect the medical records of the pet in a process 236.

In an embodiment, the data processor 110 may collect the medical records of the pet input by a veterinarian and stored in the third server 180.

For example, the medical records may include a site and a date of medical treatment for the pet, and a medical examination result of disease screening, blood test or imaging test according to the medical treatment.

Herein, the processes 230, 232, 234 and 236 are described as sequential, but may be performed in parallel, or the order of the processes may be changed or modified.

The data processor 110 according to an embodiment may calculate the level of conscientiousness of the pet owner based on the maintenance information in a process 250.

In an embodiment, the data processor 110 may generate or extract at least one index by using the maintenance information, and may calculate the level of conscientiousness of the pet owner by using the index.

For example, the data processor 110 may generate an index related to the frequency of maintenance by the pet owner based on cleaning information, consumable replacement information, water supply information and/or feed supply information of the IoT devices 120, 130, 140 and 150 included in the maintenance information. As the index related to the frequency of maintenance increases, the data processor 110 may calculate the level of conscientiousness of the pet owner to be high. On the contrary, as the index related to the frequency of maintenance decreases, the data processor 110 may calculate the level of conscientiousness of the pet owner to be low.

The data processor 110 according to an embodiment may calculate the level of conscientiousness of the pet owner further based on the input information input into an application corresponding to at least one of the IoT devices 120, 130, 140 and 150. In an embodiment, the data processor 110 may calculate the level of conscientiousness of the pet owner based on the maintenance information and the input information.

For example, the data processor 110 may generate an index related to the frequency of input or care of the pet based on the input information input into the application corresponding to the at least one of the IoT devices 120, 130, 140 and 150. As the index related to the frequency of input or care of the pet increases, the data processor 110 may calculate the level of conscientiousness of the pet owner to be high. On the contrary, as the index related to the frequency of input or care of the pet decreases, the data processor 110 may calculate the level of conscientiousness of the pet owner to be low.

The data processor 110 according to an embodiment may calculate the level of conscientiousness of the pet owner further based on the purchase information of the items for the pet. In an embodiment, the data processor 110 may calculate the level of conscientiousness of the pet owner based on the maintenance information and the purchase information of the items for the pet.

For example, the data processor 110 may generate an index related to the frequency of purchase or the amount of purchase based on the purchase information. As the index related to the frequency of purchase or the amount of purchase increases, the data processor 110 may calculate the level of conscientiousness of the pet owner to be high. On the contrary, as the index related to the frequency of purchase or the amount of purchase decreases, the data processor 110 may calculate the level of conscientiousness of the pet owner to be low.

In an embodiment, the data processor 110 may be trained with the relationship between the maintenance information, the input information and/or the purchase information and the level of conscientiousness of the pet owner by a learning model. In an embodiment, the data processor 110 may calculate the level of conscientiousness of the pet owner depending on the maintenance information, the input information and/or the purchase information based on a pre-trained learning model.

The data processor 110 according to an embodiment may calculate the level of health of the pet based on the usage information of at least one of the IoT devices 120, 130, 140 and 150 in a process 260.

In an embodiment, the data processor 110 may generate an index related to the health of the pet based on the usage information of at least one of the IoT devices 120, 130, 140 and 150. The data processor 110 may calculate the level of health of the pet based on the generated index.

In an embodiment, the data processor 110 may be trained with the relationship between the usage information of at least one of the IoT devices 120, 130, 140 and 150 and the level of health of the pet by a learning model. In an embodiment, the data processor 110 may calculate the level of health of the pet depending on the usage information of at least one of the IoT devices 120, 130, 140 and 150 based on a pre-trained learning model.

Herein, the processes 250 and 260 are described as sequential, but may be performed in parallel, or the order of the processes may be changed or modified.

The data processor 110 according to an embodiment may determine an insurance rate for the pet based on the level of conscientiousness of the pet owner and the level of health of the pet in a process 270.

In an embodiment, the data processor 110 may determine the insurance rate for the pet according to the level of conscientiousness of the pet owner and the level of health of the pet based on a predetermined equation.

A method for determining an insurance rate for a pet by the above-described data processor 110 by using at least one of the IoT devices 120, 130, 140 and 150 used in the daily life of the pet can be implemented as a computer program stored in a computer readable storage medium to be executed by a computer or a storage medium including instructions executable by a computer. Also, the method for determining an insurance rate for a pet by the above-described data processor 110 by using at least one of the IoT devices 120, 130, 140 and 150 used in the daily life of the pet can be implemented as a computer program stored in a computer readable storage medium to be executed by a computer.

A computer-readable recording medium may be any available medium that can be accessed by a computer, including volatile and non-volatile media, removable and non-removable media. Additionally, a computer-readable recording medium can include computer storage media. Computer storage media may be implemented with any method or technology for storage of information, such as computer-readable instructions, data structures, program modules, or other data. These storage media can be volatile or non-volatile, removable or non-removable.

Functions implemented by components disclosed herein in the specification can be performed by processing circuitry including general-purpose processors, specific-purpose processors, integrated circuits, ASICs (Application Specific Integrated Circuits), CPUs (Central Processing Units), circuits, and/or combinations thereof. The processors include transistors or other circuitry and are considered circuits or processing circuitry. The processor may be a programmed processor executing programs stored in memory.

In the specification, circuits, components, units, or means are hardware or hardware executing the functions disclosed in the specification. The hardware can be any hardware known in the specification or any hardware known to perform the functions disclosed in the specification.

If the hardware is considered to be a circuit type processor, the circuit, the component, the means, or the unit is a combination of hardware and or software used to configure the hardware and/or processor.

The above description of the present disclosure is provided for the purpose of illustration, and it would be understood by a person with ordinary skill in the art to which the present invention belongs that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner, likewise, components described to be distributed can be implemented in a combined manner.

The scope of the present disclosure is defined by the following claims rather than by the detailed description of the embodiment, and it should be understood that all modifications and embodiments conceived from the meaning and scope of the claims and their equivalents are included in the scope of the present disclosure.

We claim:

1. A method for determining an insurance rate for a pet by using at least one internet of things (IoT) device used in daily life of the pet, comprising:
collecting basic information of the pet;
collecting maintenance information about maintenance of the at least one IoT device by a pet owner and usage information about usage of the at least one IoT device by the pet;
calculating a level of conscientiousness of the pet owner based on the maintenance information;
calculating a level of health of the pet based on the usage information; and
determining the insurance rate for the pet based on the level of conscientiousness of the pet owner and the level of health of the pet.

2. The method for determining an insurance rate for a pet of claim 1,
wherein the at least one IoT device includes at least one of a toilet, a tracker, a feeder, or a water dispenser for use by the pet.

3. The method for determining an insurance rate for a pet of claim 1,
wherein the maintenance information includes at least one of cleaning information, consumable replacement information, water supply information, or feed supply information of the at least one IoT device.

4. The method for determining an insurance rate for a pet of claim 1, further comprising:
collecting input information input by the pet owner into an application corresponding to the at least one IoT device,
wherein the calculating the level of conscientiousness of the pet owner includes calculating the level of conscientiousness of the pet owner further based on the collected input information.

5. The method for determining an insurance rate for a pet of claim 1, further comprising:
collecting purchase information of items for the pet,
wherein the calculating the level of conscientiousness of the pet owner includes calculating the level of conscientiousness of the pet owner further based on the collected purchase information.

6. The method for determining an insurance rate for a pet of claim 5,
wherein the collecting the purchase information of the items for the pet includes collecting the purchase information from payment history at an exclusive shopping mall for the pet or of an exclusive payment card.

7. The method for determining an insurance rate for a pet of claim 1,
wherein the usage information includes at least one of usage of a toilet, amount of activity, amount of feed intake, or amount of water intake of the pet.

8. The method for determining an insurance rate for a pet of claim 1, further comprising:
collecting medical records of the pet,
wherein the calculating the level of health of the pet further includes calculating the level of health of the pet further based on the collected medical records.

9. The method for determining an insurance rate for a pet of claim 1,
wherein the basic information includes at least one of a breed, a photo, biometric data, an age, a height, a body weight, or initial health conditions of the pet.

10. The method for determining an insurance rate for a pet of claim 1,
wherein the determining the insurance rate for the pet includes determining the insurance rate for the pet according to the level of conscientiousness of the pet owner and the level of health of the pet based on a predetermined equation.

11. A system for determining an insurance rate for a pet by using at least one internet of things (IoT) device used in daily life of the pet, comprising:
an IoT device; and
a data processor,
wherein the data processor includes:
a collection unit configured to collect basic information of the pet, maintenance information about maintenance of the at least one IoT device by a pet owner, and usage information about usage of the at least one IoT device by the pet;
a calculation unit configured to calculate a level of conscientiousness of the pet owner based on the maintenance information and calculate a level of health of the pet based on the usage information; and
a determination unit configured to determine the insurance rate for the pet based on the level of conscientiousness of the pet owner and the level of health of the pet.

12. The system for determining an insurance rate for a pet of claim 11, further comprising:
a first server configured to store the basic information of the pet and input information input by the pet owner into an application corresponding to at least one IoT device,
wherein the collection unit is configured to collect the basic information of the pet and the input information from the first server, and
the calculation unit is configured to calculate the level of conscientiousness of the pet owner further based on the collected input information.

13. The system for determining an insurance rate for a pet of claim 11, further comprising:
a second server configured to store purchase information of items for the pet,
wherein the collection unit is configured to collect the purchase information from the second server, and
the calculation unit is configured to calculate the level of conscientiousness of the pet owner further based on the collected purchase information.

14. The system for determining an insurance rate for a pet of claim 11, further comprising:
a third server configured to store medical records of the pet,
wherein the collection unit is configured to collect the medical records from the third server, and
the calculation unit is configured to calculate the level of health of the pet further based on the collected medical records.

15. A non-transitory computer readable storage medium storing a computer program including a sequence of instructions for determining an insurance rate for a pet by using at least one internet of things (IoT) device used in daily life of the pet, which when executed by a computing device, causes the computing device to:
collect basic information of the pet;
collect maintenance information about maintenance of the at least one IoT device by a pet owner and usage information about usage of the at least one IoT device by the pet;
calculate a level of conscientiousness of the pet owner based on the maintenance information;
calculate a level of health of the pet based on the usage information; and
determine the insurance rate for the pet based on the level of conscientiousness of the pet owner and the level of health of the pet.
